(19) United States
(12) Patent Application Publication
(10) Pub. No.: US 2024/0175641 A1
(43) Pub. Date: May 30, 2024

(54) DEVICE AND METHOD FOR CONTROLLING THE TEMPERATURE OF AN ITEM TO BE TEMPERATURE CONTROLLED

(71) Applicant: Barkey GmbH & Co. KG, Leopoldshoehe (DE)

(72) Inventor: Harry Rempel, Leopoldshoehe (DE)

(21) Appl. No.: 18/282,752

(22) PCT Filed: Mar. 4, 2022
(86) PCT No.: PCT/DE2022/100173

§ 371 Date: Sep. 18, 2023

(30) Foreign Application Priority Data
Mar. 22, 2021 (DE) 10 2021 106 966.1

(51) Int. Cl. F28F 1/00; F28D 7/00; F28D 7/10; F28F 9/02

(52) U.S. Cl. CPC F28F 1/006; F28D 7/0016; F28D 7/106; F28F 9/0263; F28F 2210/10

(57) ABSTRACT

A device and method for controlling the temperature of an item to be temperature controlled in a simple manner without a fixing system for fixing a temperature control body to the item to be temperature controlled being additionally necessary. The temperature control body is an inflatable body having a volume which is changeable such that the inflatable body, in the working position, rests against the circumferential wall of the item to be temperature controlled when the inflatable body is in an inflated state with an increased volumetric expansion.

The invention relates to a device for controlling the temperature of an item to be temperature controlled, comprising a temperature control body which is in a working position by resting it against a contacting portion facing the item to be temperature controlled at least in regions form-fittingly arrangeable at a circumferential wall of the item to be temperature controlled and comprising temperature control means such that the item to be temperature controlled is temperature controllable to a predetermined temperature in the working position of the temperature control body.

The invention further relates to a method for controlling the temperature of an item to be temperature controlled, wherein a temperature control body is in a working position rested with a contacting portion for thermal contact against a circumferential wall of the item to be temperature controlled.

A device for controlling the temperature of a bag filled with plasma or blood material is known from WO 2019/223828 A1 in which the bag is placed between two heating elements. The device further includes an actuator for rhythmically moving the bag such that the in particular thawing process of the bag can be accelerated. The known temperature control device had fundamentally proved successful. However, it is not usable for controlling the temperature of stem cells or the like, which are contained in a preferably cylindrical container.

A temperature control device is known from DE 2 743 919 A1 which comprises a flexibly deformable temperature control body which, in a working position, form-fittingly surrounds a wall of the object to be temperature controlled. The temperature control body is designed to be a cuff which is attached to the object via a hook-and-loop fastening connection, wherein a first portion facing the object rests against the wall of the object as a contacting portion. A cooling system which is partially integrated into the temperature control body is provided as a temperature control means. A drawback of the known temperature control device is that, due to the integration of the cooling system into the cuff, the temperature control device has a substantial weight such that an additional fixing system, for example a hook-and-loop fastener, is always necessary. Furthermore, the use of the known temperature control device is limited to certain diameters of the container.

It is therefore the object of the present invention to provide a device and a method for controlling the temperature of an item to be temperature controlled such that an item to be temperature controlled can be temperature controlled in a simple manner without a fixing system for fixing the temperature control body to the item to be temperature controlled being additionally necessary.

To achieve this object, the invention, in combination with the preamble of claim 1, is characterized in that the temperature control body is designed to be an inflatable body having a volume which is changeable such that the inflatable body, in the working position, rests against the circumferential wall of the item to be temperature controlled when the inflatable body is in an inflated state with an increased volumetric expansion.

According to the invention, an inflatable body is provided which has a volume which, in a working position, is enlarged such that it rests against the wall of the item to be temperature controlled with its contacting portion. Advantageously, the inflatable body, in the working position, surrounds the item to be temperature controlled and can temperature control the same to a predetermined temperature. The inflatable body fulfills two functions. Firstly, it serves to thermally contact the temperature control body on the item to be temperature controlled such that a fluid present in the inflatable body can serve to transfer heat with the item to be temperature controlled. Secondly, the form-fitting surrounding or enclosing of the item to be temperature controlled allows for a securing of the temperature control body to the item to be temperature controlled. An additional fixing means is not necessary. Alternatively or additionally, the pressure of the fluid arranged in the temperature control body can be increased such that the temperature control body is form-fittingly and/or force-fittingly held against the item to be temperature controlled.

According to a further development of the invention, the inflatable body is coupled with an inflation generator which applies a pressure to the fluid and thus ensures a full-surface contacting of the contacting portion of the inflatable body against the item to be temperature control. Preferably, the inflation generator is arranged outside of the inflatable body such that its weight need not be considered in the design of the temperature control device.

According to a further development of the invention, the pressurized fluid is designed to be a temperature control fluid which, by means of the temperature control means, for example a heating and/or cooling system, is set to a predetermined temperature. Thus, the pressurized temperature control fluid enables thermal contacting of the item to be temperature controlled, wherein for heating, the heat of the temperature control fluid is transferred to the item to be temperature controlled and for cooling, the temperature of the item to be temperature controlled is transferred to the temperature control body. When the temperature control fluid is guided to and from the temperature control body at a certain flow rate, a planned temperature control transfer (heat or cold transfer) can occur on the item to be temperature controlled.

According to a further development of the invention, at least the contacting portion of the inflatable body is formed from a highly flexible material such that this portion elastically stretches during the inflation of the inflatable body. The associated surface expansion of the contacting portion is used to surround the item to be temperature controlled in a desired circumferential angle region. Advantageously, in this way an item to be temperature controlled in particular can be surrounded which does not have a regular or uniform contour. This way, not only a cylindrical item to be temperature controlled can be surrounded, but also an item to be temperature controlled which has a wavy or creased circumferential surface.

According to a further development of the invention, a heating system and/or a cooling system of the invention is arranged outside of the inflatable body. In this way, the pressurized fluid (pressure medium) must only operate with such a force onto the wall of the item to be temperature controlled as is necessary to hold the weight of the temperature control body on the item to be temperature controlled.

According to a further development of the invention, the inflatable body has, in addition to the first contacting portion which forms a first portion, a second portion designed to be a carrier portion with which the contacting portion is connected. The contacting portion allows for mechanical protection of the inflatable body, because the contacting portion made with flexible material is arranged between the wall of the item to be temperature controlled and the carrier portion.

According to a further development of the invention, the carrier portion can be designed to be a hollow cylinder and/or curved. The carrier portion can be designed to adapt to the contour of the circumferential wall of the item to be temperature controlled. Through the pressurization of the inflatable body, the thermal contacting of said inflatable body against the item to be temperature controlled as well as the securing of the same at the item to be temperature controlled occurs. Owing to the contour-following design of the carrier portion, the item to be temperature controlled can be surrounded in a reflex circumferential angle region such that an optimal temperature control of the item to be temperature controlled is ensured.

According to a further development of the invention, the carrier portion of the inflatable body is designed to be flat. For completely surrounding the item to be temperature controlled, two inflatable bodies are arranged opposite from the item to be temperature controlled, wherein, in the inflated state, the contacting portions of the respective inflatable bodies are surrounded by the wall of the item to be temperature controlled in an obtuse circumferential angle.

To achieve the object, the invention has the features of claim 13.

The advantage of the method according to the invention consists therein that, through the pressurization of the inflatable body, on the one hand a temperature control body is secured at an item to be temperature controlled and, on the other hand, through the thermal contacting of a contacting portion of the inflatable body associated therewith, a heat transfer occurs from the inflatable body to the item to be temperature controlled or vice versa.

If the inflatable body has an inlet opening and a specific outlet opening, the fluid can consequently flow through the inflatable body at a preferably constant volumetric flow and can consistently provide or remove the necessary heat.

According to a further development of the invention, the pressurized fluid is pressurized with a constant pressure such that the heat transfer can be chosen depending on the selected pressure. Advantageously, a simple heat transfer between the temperature control body and the item to be temperature controlled can occur in this way.

In the following, exemplary embodiments of the invention are explained in more detail with reference to the drawings.

Therein:

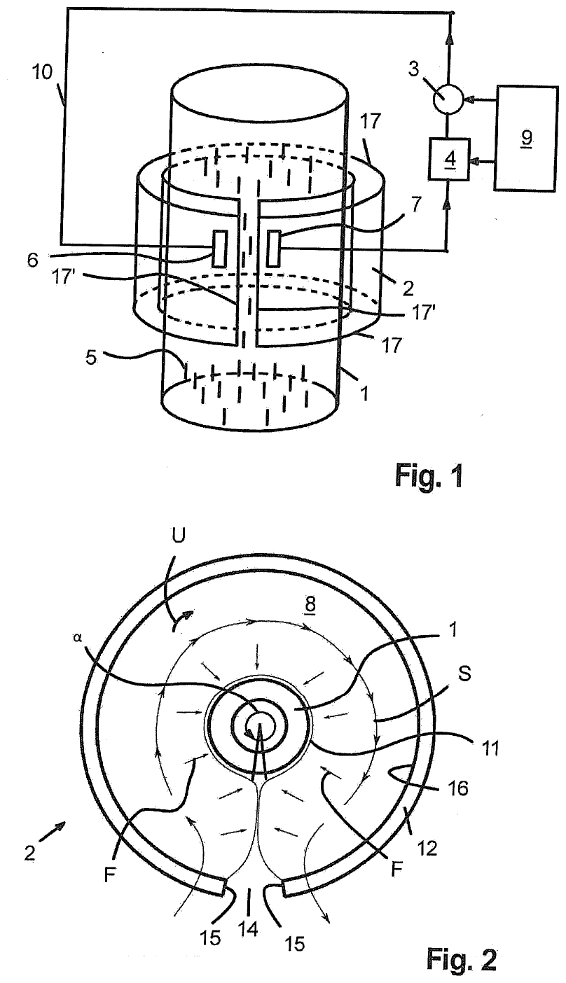

FIG. 1 shows a schematic representation of a temperature control device in a working position of the same according to a first embodiment of the invention,

FIG. 2 shows a schematic cross-section through a temperature control body of the device according to FIG. 1 in the working position of the same,

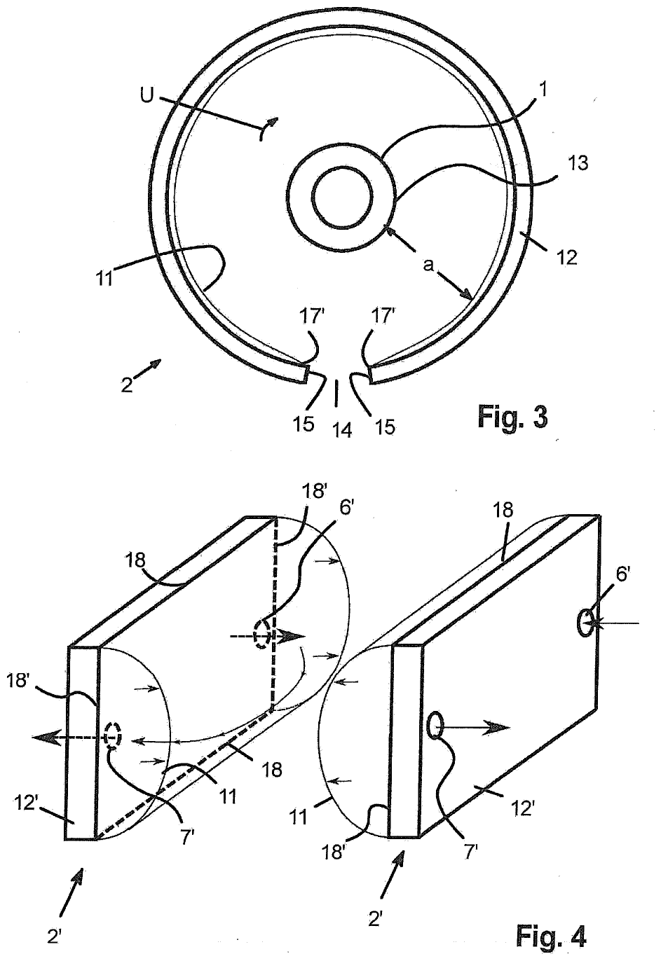

FIG. 3 shows a cross-section through the temperature control body according to FIG. 2 in a non-working position of the same,

FIG. 4 shows a perspective representation of a temperature control body according to a second embodiment,

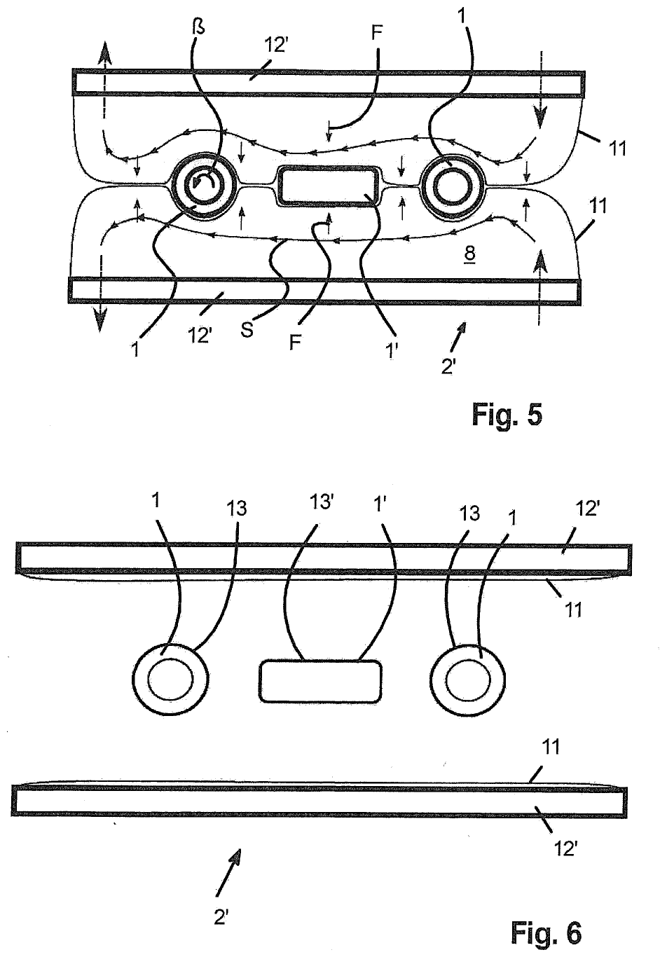

FIG. 5 shows a cross-section through the temperature control body according to FIG. 4 in the working position, and

FIG. 6 shows a cross-section through the temperature control body according to FIG. 4 in the non-working position.

A device for controlling the temperature of an item to be temperature controlled substantially consists of a temperature control body designed to be an inflatable body 2, an inflation generator 3, and a temperature control generator 4 designed to be a heating and/or cooling system. Together with a fluid 8, the temperature control generator 4 forms a temperature control means.

The item to be temperature controlled is preferably designed to be a container 1.

The container 1 in the present first exemplary embodiment is designed to be a hollow cylinder and contains an item 5 to be temperature controlled comprising, for example, stem cells, blood, and/or plasma. The container 1 serves to accommodate the item 5 to be temperature controlled and is preferably designed to be tubular. The container 1 can consist of a plastics material or of a glass material.

The inflatable body 2 surrounds the container 1 at least in regions and enables a heat transfer between the inflatable body 2 and the container 1. As can be seen in FIG. 1, the inflatable body 2 has an inlet opening 6 and an outlet opening 7 for a fluid 8 which flows through the inflatable body 2 according to a predetermined volumetric flow. To this end, a control unit 9 is provided by means of which the temperature control generator 4 and/or the inflation generator 3 is controllable such that the fluid 8 flows through the inflatable body 2 with a corresponding predetermined temperature and a predetermined volumetric flow. The fluid 8 thus serves as a temperature control fluid by means of which a heat transfer into the container 1 and/or a heat transfer out of the container 1 can occur.

The inflation generator 3 is preferably designed to be a pressure source by means of which the fluid 8, with an according volumetric flow, can be moved toward the inflatable body 2 and away from the same through a connection line 10. The fluid 8 can be in a gas state or a liquid state. For example, the fluid 8 can be designed to be air, water, oil or another liquid.

The inflatable body 2 has a contacting portion 11 on the side facing the container 1 and a carrier portion 12 on the side facing away from the container 1. The contacting portion 11 is rigidly connected to the carrier portion 12. The contacting portion 11 consists of a highly flexible and/or stretchable material, in particular of a flexible film, for example a silicone film. The carrier portion 12 consists of a stiff or bendable material which, however, in comparison to the contacting portion, is not stretchable. The carrier portion 12 can, for example, consist of a solid plastics material.

The contacting portion 11 is materially-fittingly or force-fittingly attached to an edge 17, 17′ of the carrier portion 12. The contacting portion 11 is attached to a ring-side edge 17 of the carrier portion 12 on the one hand and to a frontal edge 17′ of said carrier portion on the other. The attachment is continuous along the edges 17, 17′ such that, upon pressurization of the inflatable body 2, the contacting portion 11 separates from the inner side 16 of the carrier portion 12 and can be inflated by resting it against the container 1. For example, the contacting portion 11 can be connected to the edges 17, 17′ of the carrier portion 12 by bonding. Alternatively, the contacting portion 11 can also be mechanically attached to the edges 17, 17′ of the carrier portion 12, wherein the contacting portion 11, in the initial state without pressurization, preferably rests under tension against the inner side 16 of the carrier portion 12.

In an initial state (non-working state) of the inflatable body 2 in which no temperature control of the container 1 occurs, the contacting portion 11 rests loosely against an inner side 16 of the carrier portion 12 and has a small volume, cf. FIG. 3. The contacting portion 11 is located at a distance a from a circumferential wall 13 of the container 1. In the present exemplary embodiment, the circumferential wall 13 is circular in cross-section. The inflatable body 2 is in a non-working state (first state) or the contacting portion 11 is in a non-working position.

If the inflation generator 3 is switched on by means of the control unit 9, pressure is applied to the fluid 8 located in the line system and it is moved in a flow direction S. Due to the pressure increase, the inflatable body 2 inflates such that it, in a second state (working state) surrounds the circumferential wall 13 corresponding to a circumferential angle a with its contacting portion 11.

In FIG. 2, for a simplified representation, the contacting portion 11 of the inflatable body 2 is drawn spaced apart from the circumferential wall 13 of the container 1. In actuality, the contacting portion 11 surrounds the circumferential wall 13 form-fittingly and preferably additionally or alternatively force-fittingly, wherein a radial force F is applied to the container 1.

Through the controlling of the inflation generator 3, the temperature control generator 4 is also switched on, such that the churned fluid 8 is heated or cooled so that due to the thermal contacting of the contacting portion 11 at the circumferential wall 13 of the container 1, either a heat transfer into or out of the container can occur.

It is clear that the surface of the contacting portion 11 in the working state according to FIG. 2 is bigger than in the non-working state according to FIG. 3. This occurs due to the expansion of the contacting portion 11 as a result of the pressurization by the inflation generator 3. The contacting portion 11 adapts to the cylindrical contour or the shape of the container 1. If, for example, the contour of the container 1 were wavy in cross-section, the contacting portion 11 would adapt to the cylindrical base contour of the container 1, wherein it covers or contacts the elevations and the recesses of the circumferential wall 13.

The contacting portion 11 is preferably designed to be flexibly deformable and flexibly stretchable such that its surface increases.

According to an alternative embodiment of the invention, the carrier portion 12 can also be designed to be flexibly deformable and flexibly stretchable, wherein the shape changing feature of the contacting portion 11 is preferably greater than that of the carrier portion 12.

The contacting portion 11 and the carrier portion 12 are each formed flat, each with two parallel flat sides which provide the extension direction or the extension surface of the contacting portion 11 or the carrier portion 12. A wall thickness (distance of the flat sides to each other) of the contacting portion 11 of the carrier portion 12 is substantially smaller than the size of the flat sides and can preferably be in a mm range or cm range.

In the working state of the inflatable body 2 according to FIG. 2, the temperature controlled fluid 8 (temperature control fluid) is continuously guided into the inflatable body 2 through the inlet opening 6 and out through the outlet opening 7 at a constant volumetric flow. Through the continuous exchange of the temperature control fluid 8 within the inflatable body 2, the surface of the contacting portion 11 has a constant temperature by means of which the desired heat transfer can occur.

In the present exemplary embodiment, the carrier portion 12 is designed to be cylindrical with an opening 14 such that the carrier portion 12 can absorb forces through bending the carrier portion 12 during the transition from the non-working state to the working state and vice versa.

According to an alternative embodiment of the invention not depicted here, the carrier portion 12 can also take a circumferential angle of 360°. However, the carrier portion 12 must in this case have a separating wall so that a circular flow of the container 1 can occur via the inlet opening 6 and the outlet opening 7. In the present exemplary embodiment, the separating wall is formed by front walls 15 of the integrally designed carrier portion 12.

According to a further embodiment of the invention according to the FIGS. 4 to 6, an inflatable body 2′ is provided which, unlike the aforementioned inflatable body 2, has flat carrier portions 12′. The carrier portion 12′ is designed to be plate-shaped and has an inlet opening 6′ at a first front side and an outlet opening 7′ at an opposing front side. In the present exemplary embodiment, the inlet opening 6 is arranged at a first end region of the carrier portion 12′ and the outlet opening 7 is arranged in a second end region of the carrier portion 12′.

The contacting portion 11 is connected to the carrier portion 12′ at the straight edges 18, 18′ thereof, just as in the previous exemplary embodiment.

Identical structural parts or structural part functions in the different exemplary embodiments are provided with the same reference numerals.

The plate-shaped inflatable bodies 2′ are in particular suitable for a container which is not designed to be rotationally symmetrical. In the present exemplary embodiment, two cylindrical containers 1 and one container 1′ rectangular in cross-section for a plurality of containers are to be temperature controlled by means of two opposingly arranged inflatable bodies 2′. To this end, two inflatable bodies 2′ are arranged opposite each other, wherein the containers 1, l′ to be temperature controlled are positioned between the one inflatable body 2′ and the other inflatable body 2′. Upon application of pressure to the two inflatable bodies 2′ by means of the inflation generator 3 and of temperature by means of the temperature generator 4, the contacting portions 11 inflate by resting against opposing circumferential walls 13, 13′ of the containers 1, 1′. The two contacting portions 11 adapt to the contour or shape of the containers 1, 1′ and surround each of them in an obtuse angular region β. The angle β can, for example, also be 180°. In this way, surrounding the circumferential walls 13, 13′ occurs in a reflex angular region or at nearly 360°. As with the first embodiment, a pressure force F is applied during the inflation process by means of which the contacting portions 11 are pressed against the circumferential walls 13, 13′ of the containers 1, 1′. The respective inflatable bodies 2′ are flowed through via the respective inlet openings 6′ and outlet openings 7′ of the inflatable bodies 2′.

While the pressure force F according to the first embodiment is only directed in the radial direction toward a single rotationally symmetrically arranged container 1, the pressure force F in the second exemplary embodiment is directed in a substantially linear direction, wherein the pressure forces F applied by the two inflatable bodies 2′ are directed against each other. In this way, the containers 1, 1′ are nearly entirely surrounded or optimal thermal contacting occurs.

According to an embodiment of the invention not depicted, the container can also be partially surrounded in its circumferential direction U for temperature control. This may be appropriate when the heat transfer should only occur from one side of the container 1, l′.

The temperature region of the temperature control fluid 8 is preferably set in the region between 30° C. and 60° C. The expansion behavior of the contacting portion 11 can be configured such that its surface expands by 50% to 600% upon pressurization. The thus effected volume increase of the inflatable body 2, 2′ is dependent on the number of items 1, 1′ to be temperature controlled.

1. A device for controlling the temperature of an item (1, 1′) to be temperature controlled, comprising a temperature control body (2, 2′) which in a working position is adapted to rest with a contacting portion (11) facing the item (1, 1′) to be temperature controlled at least in regions form-fittingly against a circumferential wall (13, 13′) of the item (1, 1′) to be temperature controlled and comprising temperature control means such that the item (1, 1′) to be temperature controlled is temperature controllable to a predetermined temperature in the working position of the temperature control body (2, 2′), wherein the temperature control body is an inflatable body (2, 2′) having a volume which is changeable such that the inflatable body (2, 2′), in the working position, rests against the circumferential wall (13, 13′) of the item (1, 1′) to be temperature controlled when the inflatable body (2, 2′) is in an inflated state with an increased volumetric expansion.

2. The device according to claim 1, wherein the inflatable body (2, 2′) is coupled with an inflation generator (3), wherein, by means of the inflation generator (3), a fluid (8) is able to be let into the inflatable body (2, 2′) with such pressure that the inflatable body (2, 2′) rests with a surface-increased contacting portion (11) against the circumferential wall (13, 13′) of the item (1, 1′) to be temperature controlled.

3. The device according to claim 1, wherein the surface of the contacting portion (11) of the inflatable body (2, 2′) is larger in the working position than in a non-working position of the inflatable body (2, 2′) in which the inflatable body (2, 2′) is not inflated due to non-pressurization of the fluid (8).

4. The device according to claim 1, characterized in that wherein the contacting portion (11) of the inflatable body (2, 2′) consists of at least one of a highly flexible and a stretchable material.

5. The device according to claim 1, wherein the fluid is a temperature control fluid (8) which has a predetermined temperature.

6. The device according to claim 1, wherein the temperature control means are at least one of a heating system and a cooling system.

7. The device according to claim 6, wherein the heating system or cooling system is arranged outside of the inflatable body (2, 2′).

8. The device according to claim 1, wherein a second portion of the inflatable body (2, 2′) which is connected to the contacting portion (11) is a carrier portion (12, 12′), the form of which is independent of the pressure applied to the inflatable body (2, 2′).

9. The device according to claim 1, wherein the carrier portion (12, 12′) is material-fittingly or force-fittingly connected to the contacting portion (11) at its edges (17, 17′, 18, 18′).

10. The device according to claim 1, wherein the carrier portion (12, 12′) of the inflatable body (2, 2′) has an inlet opening (6, 6′) and an outlet opening (7, 7′) for the fluid (8).

11. The device according to claim 1, wherein the carrier portion (12) is at least one of a hollow cylinder and a curved shape, wherein, in the working position, the contacting portion (11) surrounds the item (1) to be temperature controlled at least in a reflex circumferential angle region (a).

12. The device according to claim 1, wherein the carrier portion (12′) is designed to be flat and wherein two inflatable bodies (2′) with contacting portions (11) arranged turned toward each other are provided, between which the item (1, 1′) to be temperature controlled is arranged, wherein, in the working position, the contacting portions (11) of the two opposingly arranged inflatable bodies (2′) each surround the item (1, 1′) to be temperature controlled at least in an obtuse circumferential angle region.

13. A method for controlling the temperature of an item (1, 1′) to be temperature controlled, wherein a temperature control body (2, 2′) in a working position rests with a contacting portion (11) for thermal contact against a circumferential wall (13, 13′) of the item (1, 1′) to be temperature controlled, comprising filling the temperature control body (2, 2′) with a pressurized fluid (8) having a predetermined temperature in a first state such that the temperature control body (2, 2′) rests with the contacting portion (11) against the item (1, 1′) to be temperature controlled and bringing the temperature control body (2, 2′), by non-pressurization of the fluid (8), into a non-working state in which the contacting portion (11) of the inflatable body (2, 2′) is spaced apart from the circumferential wall (13, 13′) of the item (1, 1′) to be temperature controlled.

14. The method according to claim 13, wherein the fluid (8) is pressurized with a constant pressure.

15. The method according to claim 13, wherein the fluid (8) is guided through the inflatable body (2, 2′) with a constant volumetric flow.

16. The device according to claim 1, wherein the highly flexible or stretchable material is a film.

17. The device according to claim 1, wherein a second portion of the inflatable body (2, 2′) which is connected to the contacting portion (11) is a carrier portion (12, 12′), the form of which is rigid.
